(19) United States
(12) Patent Application Publication
(10) Pub. No.: US 2021/0137358 A1
(43) Pub. Date: May 13, 2021

(54) ENDOSCOPE OBJECTIVE OPTICAL SYSTEM, IMAGE PICKUP APPARATUS AND ENDOSCOPE

(71) Applicant: OLYMPUS CORPORATION, Tokyo (JP)

(72) Inventor: Yosuke EGUCHI, Tokyo (JP)

(73) Assignee: OLYMPUS CORPORATION, Tokyo (JP)

(21) Appl. No.: 17/038,202

(22) Filed: Sep. 30, 2020

Related U.S. Application Data
(63) Continuation of application No. PCT/JP2019/007467, filed on Feb. 27, 2019.

(30) Foreign Application Priority Data
Apr. 11, 2018 (JP) 2018-076378

(51) Int. Cl. A61B 1/00

(52) U.S. Cl. CPC A61B 1/00096; A61B 1/00188; A61B 1/00009

(57) ABSTRACT

An objective optical system for an endoscope includes, in order from an object side: a first group having a positive refractive power; a second group having a negative refractive power; and a third group having a positive refractive power. At least the second group is moved along an optical axis to change magnification and perform focusing under a normal observation state and a magnified observation state, the first group includes, in order from the object side: a plano-concave negative lens with a flat surface directed to the object side; and two cemented lenses, and the following conditional expression (1) is satisfied:

−3.6<f1/fz1<−2 (1)

where f1 denotes a focal length of the plano-concave negative lens, and fz1 denotes a focal length of the entire objective optical system for an endoscope in the normal observation state.

### CROSS-REFERENCE TO RELATED APPLICATION

The present application is a continuation application of PCT/JP2019/007467 filed on Feb. 27, 2019 which is based upon and claims the benefit of priority from Japanese Patent Application No. 2018-076378 filed on Apr. 11, 2018; the entire contents of which are incorporated herein by reference.

### BACKGROUND

### Technical Field

The present disclosure relates to an objective optical system for an endoscope, an image pickup apparatus, and an endoscope.

### Description of the Related Art

In recent years, in medical endoscopes, magnifying endoscopes have been used to precisely diagnose a lesion site. Magnifying observation of a subject enables observation of a mucosal pattern and a vessel pattern, and therefore, magnifying endoscopes are used in precise diagnostics. Endoscopic images are required to have high resolution in order to improve diagnostic accuracy. For this reason, image sensors with a large number of pixels have begun to be adopted. Not only magnifying endoscopes but also ordinary endoscopes are desired to decrease a diameter in order to reduce the pain that patients feel.

Exemplary objective optical systems for such a magnifying endoscope have been proposed in the following six patent documents: Japanese Patent Application Publication No. 2009-294496; Japanese Patent Application Publication No. 2007-260305; Japanese Patent Application Publication No. 2008-107391; Japanese Patent Application Publication No. 2001-91832; Japanese Patent Application Publication No. H11-316339; and Japanese Patent No. 5985133, for example. The objective optical systems disclosed in these patent documents have a configuration with three groups of positive, negative, and positive, and perform focusing with the second group movable along an optical axis.

### SUMMARY

An objective optical system for an endoscope according to at least some embodiments in the present disclosure includes, in order from an object side:

a first group having a positive refractive power;

a second group having a negative refractive power; and

a third group having a positive refractive power, wherein

at least the second group is moved along an optical axis to change magnification and perform focusing under a normal observation state and a magnified observation state,

the first group includes, in order from the object side:

a plano-concave negative lens with a flat surface directed to the object side; and

two cemented lenses, and

the following conditional expression (1) is satisfied:

−3.6<f1/fz1<−2 (1)

where

f1 denotes a focal length of the plano-concave negative lens, and

fz1 denotes a focal length of the entire objective optical system for an endoscope in the normal observation state.

An image pickup apparatus according to at least some embodiments in the present disclosure includes the aforementioned objective optical system for an endoscope.

An endoscope according to at least some embodiments in the present disclosure includes the aforementioned objective optical system for an endoscope.

### BRIEF DESCRIPTION OF THE DRAWINGS

FIG. 1A is a sectional configuration view of a lens of an objective optical system for an endoscope in a normal observation state according to an embodiment. FIG. 1B is a sectional configuration view of the lens of the objective optical system for an endoscope in a magnified observation state according to the embodiment;

FIG. 2A is a sectional configuration view of a lens of an objective optical system for an endoscope in the normal observation state according to Example 1. FIG. 2B is a sectional configuration view of the lens of the objective optical system for an endoscope in the magnified observation state according to Example 1;

FIG. 3A illustrates spherical aberration (SA) in the normal observation state, FIG. 3B illustrates astigmatism (AS) in the normal observation state, FIG. 3C illustrates distortion (DT) in the normal observation state, and FIG. 3D illustrates a chromatic aberration of magnification (CC) in the normal observation state, for the objective optical system for an endoscope according to Example 1. FIG. 3E illustrates spherical aberration (SA) in the magnified observation state, FIG. 3F illustrates astigmatism (AS) in the magnified observation state, FIG. 3G illustrates distortion (DT) in the magnified observation state, and FIG. 3H illustrates a chromatic aberration of magnification (CC) in the magnified observation state, for the objective optical system for an endoscope according to Example 1;

FIG. 4A is a sectional configuration view of a lens of an objective optical system for an endoscope in the normal observation state according to Example 2. FIG. 4B is a sectional configuration view of the lens of the objective optical system for an endoscope in the magnified observation state according to Example 2;

FIG. 5A illustrates spherical aberration (SA) in the normal observation state, FIG. 5B illustrates astigmatism (AS) in the normal observation state, FIG. 5C illustrates distortion (DT) in the normal observation state, and FIG. 5D illustrates a chromatic aberration of magnification (CC) in the normal observation state, for the objective optical system for an endoscope according to Example 2. FIG. 5E illustrates spherical aberration (SA) in the magnified observation state, FIG. 5F illustrates astigmatism (AS) in the magnified observation state, FIG. 5G illustrates distortion (DT) in the magnified observation state, and FIG. 5H illustrates a chromatic aberration of magnification (CC) in the magnified observation state, for the objective optical system for an endoscope according to Example 2;

FIG. 6A is a sectional configuration view of a lens of an objective optical system for an endoscope in the normal observation state according to Example 3. FIG. 6B is a sectional configuration view of the lens of the objective optical system for an endoscope in the magnified observation state according to Example 3;

FIG. 7A illustrates spherical aberration (SA) in the normal observation state, FIG. 7B illustrates astigmatism (AS) in the normal observation state, FIG. 7C illustrates distortion (DT) in the normal observation state, and FIG. 7D illustrates a chromatic aberration of magnification (CC) in the normal observation state, for the objective optical system for an endoscope according to Example 3. FIG. 7E illustrates spherical aberration (SA) in the magnified observation state, FIG. 7F illustrates astigmatism (AS) in the magnified observation state, FIG. 7G illustrates distortion (DT) in the magnified observation state, and FIG. 7H illustrates a chromatic aberration of magnification (CC) in the magnified observation state, for the objective optical system for an endoscope according to Example 3;

FIG. 8A is a sectional configuration view of a lens of an objective optical system for an endoscope in the normal observation state according to Example 4. FIG. 8B is a sectional configuration view of the lens of the objective optical system for an endoscope in the magnified observation state according to Example 4;

FIG. 9A illustrates spherical aberration (SA) in the normal observation state, FIG. 9B illustrates astigmatism (AS) in the normal observation state, FIG. 9C illustrates distortion (DT) in the normal observation state, and FIG. 9D illustrates a chromatic aberration of magnification (CC) in the normal observation state, for the objective optical system for an endoscope according to Example 4. FIG. 9E illustrates spherical aberration (SA) in the magnified observation state, FIG. 9F illustrates astigmatism (AS) in the magnified observation state, FIG. 9G illustrates distortion (DT) in the magnified observation state, and FIG. 9H illustrates a chromatic aberration of magnification (CC) in the magnified observation state, for the objective optical system for an endoscope according to Example 4;

FIG. 10A is a sectional configuration view of a lens of an objective optical system for an endoscope in the normal observation state according to Example 5. FIG. 10B is a sectional configuration view of the lens of the objective optical system for an endoscope in the magnified observation state according to Example 5;

FIG. 11A illustrates spherical aberration (SA) in the normal observation state, FIG. 11B illustrates astigmatism (AS) in the normal observation state, FIG. 11C illustrates distortion (DT) in the normal observation state, and FIG. 11D illustrates a chromatic aberration of magnification (CC) in the normal observation state, for the objective optical system for an endoscope according to Example 5. FIG. 11E illustrates spherical aberration (SA) in the magnified observation state, FIG. 11F illustrates astigmatism (AS) in the magnified observation state, FIG. 11G illustrates distortion (DT) in the magnified observation state, and FIG. 11H illustrates a chromatic aberration of magnification (CC) in the magnified observation state, for the objective optical system for an endoscope according to Example 5;

FIG. 12A is a sectional configuration view of a lens of an objective optical system for an endoscope in the normal observation state according to Example 6. FIG. 12B is a sectional configuration view of the lens of the objective optical system for an endoscope in the magnified observation state according to Example 6;

FIG. 13A illustrates spherical aberration (SA) in the normal observation state, FIG. 13B illustrates astigmatism (AS) in the normal observation state, FIG. 13C illustrates distortion (DT) in the normal observation state, and FIG. 13D illustrates a chromatic aberration of magnification (CC) in the normal observation state, for the objective optical system for an endoscope according to Example 6. FIG. 13E illustrates spherical aberration (SA) in the magnified observation state, FIG. 13F illustrates astigmatism (AS) in the magnified observation state, FIG. 13G illustrates distortion (DT) in the magnified observation state, and FIG. 13H illustrates a chromatic aberration of magnification (CC) in the magnified observation state, for the objective optical system for an endoscope according to Example 6;

FIG. 14A is a sectional configuration view of a lens of an objective optical system for an endoscope in the normal observation state according to Example 7. FIG. 14B is a sectional configuration view of the lens of the objective optical system for an endoscope in the magnified observation state according to Example 7;

FIG. 15A illustrates spherical aberration (SA) in the normal observation state, FIG. 15B illustrates astigmatism (AS) in the normal observation state, FIG. 15C illustrates distortion (DT) in the normal observation state, and FIG. 15D illustrates a chromatic aberration of magnification (CC) in the normal observation state, for the objective optical system for an endoscope according to Example 7. FIG. 15E illustrates spherical aberration (SA) in the magnified observation state, FIG. 15F illustrates astigmatism (AS) in the magnified observation state, FIG. 15G illustrates distortion (DT) in the magnified observation state, and FIG. 15H illustrates a chromatic aberration of magnification (CC) in the magnified observation state, for the objective optical system for an endoscope according to Example 7;

FIG. 16A is a sectional configuration view of a lens of an objective optical system for an endoscope in the normal observation state according to Example 8. FIG. 16B is a sectional configuration view of the lens of the objective optical system for an endoscope in the magnified observation state according to Example 8; and

FIG. 17A illustrates spherical aberration (SA) in the normal observation state, FIG. 17B illustrates astigmatism (AS) in the normal observation state, FIG. 17C illustrates distortion (DT) in the normal observation state, and FIG. 17D illustrates a chromatic aberration of magnification (CC) in the normal observation state, for the objective optical system for an endoscope according to Example 8. FIG. 17E illustrates spherical aberration (SA) in the magnified observation state, FIG. 17F illustrates astigmatism (AS) in the magnified observation state, FIG. 17G illustrates distortion (DT) in the magnified observation state, and FIG. 17H illustrates a chromatic aberration of magnification (CC) in the magnified observation state, for the objective optical system for an endoscope according to Example 8.

### DETAILED DESCRIPTION

The reasons for adopting the aforementioned configuration for the objective optical system for an endoscope, the image pickup apparatus, and the endoscope according to the present embodiment and the effects thereof will be explained hereinafter. The present disclosure is not limited to the following embodiment.

### Embodiment

FIG. 1A is a sectional configuration view of a lens of an objective optical system for an endoscope in a normal observation state according to an embodiment. FIG. 1B is a sectional configuration view of the lens of the objective optical system for an endoscope in a magnified observation state according to the embodiment.

An objective optical system for an endoscope according to the embodiment includes, in order from an object side:

a first group having a positive refractive power G1;

a second group having a negative refractive power G2; and

a third group having a positive refractive power G3, wherein

at least the second group G2 is moved along an optical axis AX to change magnification and perform focusing under a normal observation state and a magnified observation state,

the first group G1 includes, in order from the object side:

a plano-concave negative lens L1 with a flat surface directed to the object side; and

two cemented lenses CL1 and CL2, and

the following conditional expression (1) is satisfied:

−3.6<f1/fz1<−2 (1)

where

f1 denotes a focal length of the plano-concave negative lens L1, and

fz1 denotes a focal length of the entire objective optical system for an endoscope in the normal observation state.

Hereinafter the plano-concave negative lens L1 may be referred to as the first lens L1 as needed.

The conditional expression (1) defines an appropriate ratio of f1 to fz1.

The lens configuration of the objective optical system for an endoscope is a three-group configuration including a first group having a positive refractive power G1, a second group having a negative refractive power G2, and a third group having a positive refractive power G3. By focusing with the negative group in the second group G2, it is possible to reduce the fluctuation of aberration at a time of focusing. In the objective optical system for an endoscope according to the present embodiment, a lens having a large negative refractive power is installed as the first lens L1 to make an angle of view wider. When the first lens L1 has a large negative refractive power, the position of the principal point is the side of an image plane (rear side). For this reason, it is possible to reduce the size of an objective optical system for an endoscope and secure a sufficient back focus.

The conditional expression (1) is a conditional expression for preventing the deterioration of optical performance due to a manufacturing error. In the present embodiment, the first lens L1 has a large negative refractive power, so that the objective optical system for an endoscope reduces the size and thus improves the observability, but on the other hand, because the first lens L1 has a large refractive power, if a manufacturing error has occurred, the deterioration of optical performance can be significant.

When the conditional expression (1) takes a value larger than the upper limit value thereof, the focal length f1 of the plano-concave negative lens L1 becomes excessively large. The radius of curvature of the first lens L1 becomes small, and thus the refraction power of the first lens L1 becomes large. For these reasons, dispersion of peripheral performance due to manufacturing errors in lenses becomes large.

When the conditional expression (1) takes a value smaller than the lower limit value thereof, the focal length f1 of the plano-concave negative lens L1 is excessively small. For this reason, spherical aberration, coma, and the like occur, and thus the optical performance deteriorates.

According to a preferred aspect of the present embodiment, it is preferable that the third group G3 include: a positive lens L8; a positive lens L9; and a cemented lens CL3 including a positive lens L10 and a negative lens L11, and the following conditional expressions (2) and (3) be satisfied:

−5<f5/f7<−1 (2)

−5<f6/f7<−0.3 (3)

f5 denotes a focal length of the object-side positive lens L8 of the third group G3,

f6 denotes a focal length of the image-side positive lens L9 of the third group G3, and

f7 denotes a focal length of the cemented lens CL3 of the third group G3.

The conditional expression (2) defines an appropriate ratio of f5 to f7.

The conditional expression (3) defines an appropriate ratio of f6 to f7.

In a magnifying optical system, oblique incidence characteristics of an image sensor needs to be on a minus side with respect to a plane perpendicular to the optical axis AX. If the rays are bent steeply by the positive lenses in a normal state, ray height increases near the positive lenses of the third group G3, thereby causing flare. For this reason, refractive powers need to be distributed appropriately to the positive lenses of the third group G3.

When the conditional expressions (2) and (3) take values larger than the upper limit values thereof, the refractive powers of the positive lenses L8 and L9 become excessively large and thus spherical aberration is overcorrected.

When the conditional expressions (2) and (3) take values smaller than the lower limit values thereof, the refractive powers of the positive lenses L8 and L9 becomes small and thus the oblique incidence characteristics are inclined to a plus side.

It is preferable that the following conditional expressions (2′) and (3′) be satisfied, instead of the conditional expressions (2′) and (3′):

−3<f5/f7<−1 (2′)

−3<f6/f7<−0.9 (3′)

According to a preferred aspect of the present embodiment, it is preferable that the following conditional expression (4) be satisfied:

2.1<Ls/Bk<5 (4)

Ls denotes a movement distance of the second group G2 from the normal observation state to the magnified observation state, and

Bk denotes a distance from the final surface of the objective optical system for an endoscope to an image plane I along the optical axis AX.

The conditional expression (4) is a conditional expression for setting appropriately the movement distance (optical stroke) of the second group G2 from the normal observation state to the magnified observation state, and the distance from the final surface of the objective optical system for an endoscope to the image plane I along the optical axis (back focus).

When the conditional expression (4) takes a value larger than the upper limit value thereof, a sufficient back focus is not secured, focusing cannot be adjusted, and thus a lens cannot be assembled.

When the conditional expression (4) takes a value smaller than the lower limit value thereof, the stroke becomes short, sensitivity in changing magnification becomes high, and thus the controllability of the objective optical system for an endoscope deteriorates.

It is preferable that the following conditional expression (4′) be satisfied, instead of the conditional expression (4):

2.4<Ls/Bk<4.6 (4′)

According to a preferred aspect of the present embodiment, it is preferable that the following conditional expression (5) be satisfied:

0.8<FFz3/fz3<4 (5)

FFz3 denotes a distance from the front focal point of the objective optical system for an endoscope in the magnified observation state to the surface of the objective optical system for an endoscope positioned nearest to the object (front focal point), and

fz3 denotes a focal length of the entire objective optical system for an endoscope in the magnified observation state.

The conditional expression (5) is a conditional expression for setting appropriately the magnification at the magnifying observation.

When the conditional expression (5) takes a value larger than the upper limit value thereof, FFz3 becomes excessively large, so that the magnification at the magnifying observation becomes excessively small and makes the resolution of a subject to be observed insufficient. For this reason, the observability deteriorates.

When the conditional expression (5) takes a value smaller than the lower limit value thereof, FFz3 becomes small, and thus the magnification at the magnifying observation becomes large. The distortion, however, becomes excessively large, and thus the peripheral area looks dense at the magnifying observation. For this reason, the observability deteriorates.

It is preferable that the following conditional expression (5′) be satisfied, instead of the conditional expression (5):

1.1<FFz3/fz3<3 (5′)

According to a preferred aspect of the present embodiment, it is preferable that the following conditional expression (6) be satisfied:

−6<f7/f3<−0.5 (6)

f7 denotes a focal length of the cemented lens CL3 of the third group G3, and

f3 denotes a focal length of the image-side cemented lens CL2 of the first group G1.

The conditional expression (6) is a conditional expression for satisfying the oblique incidence characteristics of the image sensor.

When the conditional expression (6) takes a value larger than the upper limit value thereof, the refractive power of f7 becomes excessively large. For this reason, the sensitivity to chromatic aberration of magnification of f7 due to a manufacturing error becomes excessively high and thus the performance deteriorates.

When the conditional expression (6) takes a value smaller than the lower limit value thereof, the refractive power of f7 becomes excessively small. For this reason, the oblique incidence characteristics of the image sensor are inclined to a plus side and thus the peripheral area of the view becomes dark.

It is preferable that the following conditional expression (6′) be satisfied, instead of the conditional expression (6):

−2<f7/f3<−0.74 (6′)

According to a preferred aspect of the present embodiment, it is preferable that the following conditional expression (7) be satisfied:

−30<f2/fz1<−1 (7)

f2 denotes a focal length of the object-side cemented lens CL1 of the first group G1, and

fz1 denotes the focal length of the entire objective optical system for an endoscope in the normal observation state.

The conditional expression (7) defines an appropriate ratio of f2 to fz1.

The cemented lens CL1 has effects of correcting chromatic aberration, and correcting curvature of field that the large refractive power of the first lens L1 generates.

When the conditional expression (7) takes a value larger than the upper limit value thereof, the focal length f2 of the object-side cemented lens CL1 becomes large and thus the distance between the sagittal image plane (S) and the meridional image plan (M) becomes large. For this reason, astigmatism is undercorrected.

When the conditional expression (7) takes a value smaller than the lower limit value thereof, the focal length f2 of the object-side cemented lens CL1 becomes small, the refractive power thereof becomes excessively small, and thus the effect of correcting chromatic aberration disappears.

It is preferable that the following conditional expression (7′) be satisfied, instead of the conditional expression (7):

−26<f2/fz1<−5 (7′)

According to a preferred aspect of the present embodiment, it is preferable that the following conditional expression (8) be satisfied:

−8<f2/f3<−1 (8)

f2 denotes the focal length of the object-side cemented lens CL1 of the first group G1, and

f3 denotes the focal length of the image-side cemented lens CL2 of the first group G1.

The conditional expression (8) is a conditional expression for processability of a lens and correcting curvature of field.

When the conditional expression (8) takes a value larger than the upper limit value thereof, the radius of curvature at the cemented surface of f2 becomes excessively tight (small), a sufficient thickness of the peripheral part of a lens cannot be secured, and thus it is not favorable for manufacturing.

When the conditional expression (8) takes a value smaller than the lower limit value thereof, the radius of curvature at the cemented surface of f2 becomes excessively gentle (large) and thus the curvature of field cannot be sufficiently corrected.

It is preferable that the following conditional expression (8′) be satisfied, instead of the conditional expression (8):

−4<f2/f3<−1.1 (8′)

According to a preferred aspect of the present embodiment, it is preferable that the third group G3 include a plano-convex positive lens L12 with a flat surface cemented to a cover glass CG and directed to the image plane and the following conditional expression (9) be satisfied. The cover glass CG is a parallel plate.

−10<f8/f1<−0.5 (9)

where

f8 denotes a focal length of the positive lens L12 cemented to the cover glass CG, and

f1 denotes a focal length of the plano-concave negative lens L1.

The conditional expression (9) is a conditional expression related to adjustment sensitivity in assembling an optical system.

When the conditional expression (9) takes a value smaller than the lower limit value thereof, f8 becomes excessively small. For this reason, the adjustment sensitivity in assembling an optical system becomes oversensitive.

When the conditional expression (9) takes a value larger than the upper limit value thereof, the refractive power of f1 becomes excessively large. For these reasons, dispersion of peripheral performance due to manufacturing errors in an optical system becomes large.

It is preferable that the following conditional expression (9′) be satisfied, instead of the conditional expression (9):

−7<f8/f1<−1.2 (9′)

According to a preferred aspect of the present embodiment, it is preferable that the cemented lens CL3 of the third group G3 include a biconcave negative lens L11, and the following conditional expression (10) be satisfied.

0.1<SF72<0.9 (10)

SF72 denotes a shaping factor of the biconcave negative lens L11. When r72 is a radius of the object-side curvature of the biconcave negative lens L11 and r73 is a radius of the image-side curvature of the biconcave negative lens L11, SF72=(r72+r73)/(r72−r73).

The conditional expression (10) is a conditional expression for satisfying the oblique incidence characteristics of the image sensor.

When the conditional expression (10) takes a value larger than the upper limit value thereof, the radius of curvature at the cemented surface of the cemented lens CL3 becomes excessively large and thus the chromatic aberration of magnification cannot be corrected.

When the conditional expression (10) takes a value smaller than the lower limit value thereof, the shaping factor becomes excessively small and thus the radius of curvature on the image plane side becomes small. For this reason, the oblique incidence characteristics of the image plane are inclined to a plus side and thus the peripheral area of the view becomes dark.

It is preferable that the following conditional expression (10′) be satisfied, instead of the conditional expression (10):

0.2<SF72<0.7 (10′)

### Example 1

An objective optical system for an endoscope according to Example 1 will be explained hereinafter.

FIG. 2A is a sectional configuration view of a lens of an objective optical system for an endoscope in the normal observation state according to Example 1. FIG. 2B is a sectional configuration view of the lens of the objective optical system for an endoscope in the magnified observation state according to Example 1.

The objective optical system for an endoscope includes, in order from an object side: a first group having a positive refractive power G1; an aperture stop S; a second group having a negative refractive power G2; and a third group having a positive refractive power G3.

The first group G1 having a positive refractive power includes, in order from the object side: a plano-concave negative lens L1 with a flat surface directed to the object side; a biconcave negative lens L2; a biconvex positive lens L3; an infrared absorbing filter F1; a plano-convex positive lens L4 with a flat surface directed to the object side; and a negative meniscus lens L5 with a convex surface directed to an image. The negative lens L2 and the positive lens L3 are cemented to forma cemented lens CL1. The positive lens L4 and the negative meniscus lens L5 are cemented to form a cemented lens CL2. The aperture stop S is disposed on the image side of the first group G1.

The second group G2 having a negative refractive power includes, in order from the object side: a plano-concave negative lens L6 with a flat surface directed to the object side; and a positive meniscus lens L7 with a convex surface directed to the object side. The negative lens L6 and the positive lens L7 are cemented. The second group G2 moves toward the image along the optical axis AX at a time of focusing from the normal observation state to the magnified observation state. r12 and r16 are virtual planes.

The third group G3 having a positive refractive power includes, in order from the object side: a biconvex positive lens L8; a plano-convex positive lens L9 with a flat surface directed to the image; a biconvex positive lens L10; a biconcave negative lens L11; and a plano-convex positive lens L12 with a flat surface directed to the image. The positive lens L10 and the negative lens L11 are cemented to form a cemented lens CL3. The positive lens L12 and a cover glass CG are cemented. The cover glass CG being a parallel plate is cemented to an image plane I that is the image pickup surface of an image sensor not illustrated.

The infrared absorbing filter F1 being a parallel plate is a filter provided with a coating for cutting a specific wavelength, for example, 1060 nm of a YAG laser, 810 nm of a semiconductor laser, or an infrared band.

FIG. 3A illustrates spherical aberration (SA) in the normal observation state, FIG. 3B illustrates astigmatism (AS) in the normal observation state, FIG. 3C illustrates distortion (DT) in the normal observation state, and FIG. 3D illustrates a chromatic aberration of magnification (CC) in the normal observation state, for the objective optical system for an endoscope according to Example 1.

FIG. 3E illustrates spherical aberration (SA) in the magnified observation state, FIG. 3F illustrates astigmatism (AS) in the magnified observation state, FIG. 3G illustrates distortion (DT) in the magnified observation state, and FIG. 3H illustrates a chromatic aberration of magnification (CC) in the magnified observation state, for the objective optical system for an endoscope according to Example 1.

These aberration diagrams illustrate respective aberrations at the wavelengths of 546.7 nm (e-line), 435.84 (g-line), 486.13 (F-line), and 656.3 nm (C-line). In the diagrams, “FIY” denotes the image height. Hereinafter, the same signs are used in the aberration diagrams.

### Example 2

An objective optical system for an endoscope according to Example 2 will be explained hereinafter.

FIG. 4A is a sectional configuration view of a lens of an objective optical system for an endoscope in the normal observation state according to Example 2. FIG. 4B is a sectional configuration view of the lens of the objective optical system for an endoscope in the magnified observation state according to Example 2.

The objective optical system for an endoscope includes, in order from an object side: a first group G1 having a positive refractive power; an aperture stop S; a second group G2 having a negative refractive power; and a third group G3 having a positive refractive power.

The first group G1 having a positive refractive power includes, in order from the object side: a plano-concave negative lens L1 with a flat surface directed to the object side; a biconcave negative lens L2; a biconvex positive lens L3; an infrared absorbing filter F1; a biconvex positive lens L4; and a negative meniscus lens L5 with a convex surface directed to an image. The negative lens L2 and the positive lens L3 are cemented to forma cemented lens CL1. The positive lens L4 and the negative meniscus lens L5 are cemented to form a cemented lens CL2. The aperture stop S is disposed on the image side of the first group G1.

The second group G2 having a negative refractive power includes, in order from the object side: a negative meniscus lens L6 with a convex surface directed to the object side; and a positive meniscus lens L7 with a convex surface directed to the object side. The negative meniscus lens L6 and the positive meniscus lens L7 are cemented. The second group G2 moves toward the image along the optical axis AX at a time of focusing from the normal observation state to the magnified observation state. r12 and r16 are virtual planes.

The third group having G3 a positive refractive power includes, in order from the object side: a biconvex positive lens L8; a biconvex positive lens L9; biconvex positive lens L10; a biconcave negative lens L11; and a plano-convex positive lens L12 with a flat surface directed to the image. The positive lens L10 and the negative lens L11 are cemented to form a cemented lens CL3. The positive lens L12 and a cover glass CG are cemented. The cover glass CG being a parallel plate is cemented to an image plane I that is the image pickup surface of an image sensor not illustrated.

The infrared absorbing filter F1 being a parallel plate is a filter provided with a coating for cutting a specific wavelength, for example, 1060 nm of a YAG laser, 810 nm of a semiconductor laser, or an infrared band.

FIG. 5A illustrates spherical aberration (SA) in the normal observation state, FIG. 5B illustrates astigmatism (AS) in the normal observation state, FIG. 5C illustrates distortion (DT) in the normal observation state, and FIG. 5D illustrates a chromatic aberration of magnification (CC) in the normal observation state, for the objective optical system for an endoscope according to Example 2.

FIG. 5E illustrates spherical aberration (SA) in the magnified observation state, FIG. 5F illustrates astigmatism (AS) in the magnified observation state, FIG. 5G illustrates distortion (DT) in the magnified observation state, and FIG. 5H illustrates a chromatic aberration of magnification (CC) in the magnified observation state, for the objective optical system for an endoscope according to Example 2.

### Example 3

An objective optical system for an endoscope according to Example 3 will be explained hereinafter.

FIG. 6A is a sectional configuration view of a lens of an objective optical system for an endoscope in the normal observation state according to Example 3. FIG. 6B is a sectional configuration view of the lens of the objective optical system for an endoscope in the magnified observation state according to Example 3.

The objective optical system for an endoscope includes, in order from an object side: a first group G1 having a positive refractive power; an aperture stop S; a second group G2 having a negative refractive power; and a third group G3 having a positive refractive power.

The first group G1 having a positive refractive power includes, in order from the object side: a plano-concave negative lens L1 with a flat surface directed to the object side; a biconcave negative lens L2; a biconvex positive lens L3; an infrared absorbing filter F1; a biconvex positive lens L4; and a negative meniscus lens L5 with a convex surface directed to an image. The negative lens L2 and the positive lens L3 are cemented to form a cemented lens CL1. The positive lens L4 and the negative meniscus lens L5 are cemented to form a cemented lens CL2. The aperture stop S is disposed on the image side of the first group G1.

The second group G2 having a negative refractive power includes, in order from the object side: a negative meniscus lens L6 with a convex surface directed to the object side; and a positive meniscus lens L7 with a convex surface directed to the object side. The negative meniscus lens L6 and the positive meniscus lens L7 are cemented. The second group G2 moves toward the image along the optical axis AX at a time of focusing from the normal observation state to the magnified observation state. r12, r16, and r24 are virtual planes.

The third group G3 having a positive refractive power includes, in order from the object side: a biconcave negative lens L8; a biconvex positive lens L9; biconvex positive lens L10; a biconcave negative lens L11; and a plano-convex positive lens L12 with a flat surface directed to the image. The positive lens L10 and the negative lens L11 are cemented to form a cemented lens CL3. The positive lens L12 and a cover glass CG are cemented. The cover glass CG being a parallel plate is cemented to an image plane I that is the image pickup surface of an image sensor not illustrated.

The infrared absorbing filter F1 being a parallel plate is a filter provided with a coating for cutting a specific wavelength, for example, 1060 nm of a YAG laser, 810 nm of a semiconductor laser, or an infrared band.

FIG. 7A illustrates spherical aberration (SA) in the normal observation state, FIG. 7B illustrates astigmatism (AS) in the normal observation state, FIG. 7C illustrates distortion (DT) in the normal observation state, and FIG. 7D illustrates a chromatic aberration of magnification (CC) in the normal observation state, for the objective optical system for an endoscope according to Example 3.

FIG. 7E illustrates spherical aberration (SA) in the magnified observation state, FIG. 7F illustrates astigmatism (AS) in the magnified observation state, FIG. 7G illustrates distortion (DT) in the magnified observation state, and FIG. 7H illustrates a chromatic aberration of magnification (CC) in the magnified observation state, for the objective optical system for an endoscope according to Example 3.

### Example 4

An objective optical system for an endoscope according to Example 4 will be explained hereinafter.

FIG. 8A is a sectional configuration view of a lens of an objective optical system for an endoscope in the normal observation state according to Example 4. FIG. 8B is a sectional configuration view of the lens of the objective optical system for an endoscope in the magnified observation state according to Example 4.

The objective optical system for an endoscope includes, in order from an object side: a first group G1 having a positive refractive power; an aperture stop S; a second group G2 having a negative refractive power; and a third group G3 having a positive refractive power.

The first group G1 having a positive refractive power includes, in order from the object side: a plano-concave negative lens L1 with a flat surface directed to the object side; a biconcave negative lens L2; a biconvex positive lens L3; an infrared absorbing filter F1; a plano-convex positive lens L4 with a flat surface directed to the object side; and a negative meniscus lens L5 with a convex surface directed to an image. The negative lens L2 and the positive lens L3 are cemented to forma cemented lens CL1. The positive lens L4 and the negative meniscus lens L5 are cemented to form a cemented lens CL2. The aperture stop S is disposed on the image side of the first group G1.

The second group G2 having a negative refractive power includes, in order from the object side: a negative meniscus lens L6 with a convex surface directed to the object side; and a positive meniscus lens L7 with a convex surface directed to the object side. The negative meniscus lens L6 and the positive meniscus lens L7 are cemented. The second group G2 moves toward the image along the optical axis AX at a time of focusing from the normal observation state to the magnified observation state. r12, r16, and r24 are virtual planes.

The third group G3 having a positive refractive power includes, in order from the object side: a biconvex positive lens L8; a plano-convex positive lens L9 with a flat surface directed to the image; a biconvex positive lens L10; a biconcave negative lens L11; and a plano-convex positive lens L12 with a flat surface directed to the image. The positive lens L10 and the negative lens L11 are cemented to form a cemented lens CL3. The positive lens L12 and a cover glass CG are cemented. The cover glass CG being a parallel plate is cemented to an image plane I that is the image pickup surface of an image sensor not illustrated.

The infrared absorbing filter F1 being a parallel plate is a filter provided with a coating for cutting a specific wavelength, for example, 1060 nm of a YAG laser, 810 nm of a semiconductor laser, or an infrared band.

FIG. 9A illustrates spherical aberration (SA) in the normal observation state, FIG. 9B illustrates astigmatism (AS) in the normal observation state, FIG. 9C illustrates distortion (DT) in the normal observation state, and FIG. 9D illustrates a chromatic aberration of magnification (CC) in the normal observation state, for the objective optical system for an endoscope according to Example 4.

FIG. 9E illustrates spherical aberration (SA) in the magnified observation state, FIG. 9F illustrates astigmatism (AS) in the magnified observation state, FIG. 9G illustrates distortion (DT) in the magnified observation state, and FIG. 9H illustrates a chromatic aberration of magnification (CC) in the magnified observation state, for the objective optical system for an endoscope according to Example 4.

### Example 5

An objective optical system for an endoscope according to Example 5 will be explained hereinafter.

FIG. 10A is a sectional configuration view of a lens of an objective optical system for an endoscope in the normal observation state according to Example 5. FIG. 10B is a sectional configuration view of the lens of the objective optical system for an endoscope in the magnified observation state according to Example 5.

The objective optical system for an endoscope includes, in order from an object side: a first group G1 having a positive refractive power; an aperture stop S; a second G2 having a negative refractive power; and a third group G3 having a positive refractive power.

The first group G1 having a positive refractive power includes, in order from the object side: a plano-concave negative lens L1 with a flat surface directed to the object side; a biconcave negative lens L2; a biconvex positive lens L3; an infrared absorbing filter F1; a biconvex positive lens L4; and a negative meniscus lens L5 with a convex surface directed to an image. The negative lens L2 and the positive lens L3 are cemented to forma cemented lens CL1. The positive lens L4 and the negative meniscus lens L5 are cemented to form a cemented lens CL2. The aperture stop S is disposed on the image side of the first group G1.

The second group G2 having a negative refractive power includes, in order from the object side: a biconcave negative lens L6; and a positive meniscus lens L7 with a convex surface directed to the object side. The negative lens L6 and the positive lens L7 are cemented. The second group G2 moves toward the image along the optical axis AX at a time of focusing from the normal observation state to the magnified observation state. r12 and r16 are virtual planes.

The third group G3 having a positive refractive power includes, in order from the object side: a biconvex positive lens L8; a plano-convex positive lens L9 with a flat surface directed to the image; a biconvex positive lens L10; a biconcave negative lens L11; and a plano-convex positive lens L12 with a flat surface directed to the image. The positive lens L10 and the negative lens L11 are cemented to form a cemented lens CL3. The cover glass CG being a parallel plate is cemented to an image plane I that is the image pickup surface of an image sensor not illustrated.

The infrared absorbing filter F1 being a parallel plate is a filter provided with a coating for cutting a specific wavelength, for example, 1060 nm of a YAG laser, 810 nm of a semiconductor laser, or an infrared band.

FIG. 11A illustrates spherical aberration (SA) in the normal observation state, FIG. 11B illustrates astigmatism (AS) in the normal observation state, FIG. 11C illustrates distortion (DT) in the normal observation state, and FIG. 11D illustrates a chromatic aberration of magnification (CC) in the normal observation state, for the objective optical system for an endoscope according to Example 5.

FIG. 11E illustrates spherical aberration (SA) in the magnified observation state, FIG. 11F illustrates astigmatism (AS) in the magnified observation state, FIG. 11G illustrates distortion (DT) in the magnified observation state, and FIG. 11H illustrates a chromatic aberration of magnification (CC) in the magnified observation state, for the objective optical system for an endoscope according to Example 5.

### Example 6

An objective optical system for an endoscope according to Example 6 will be explained hereinafter.

FIG. 12A is a sectional configuration view of a lens of an objective optical system for an endoscope in the normal observation state according to Example 6. FIG. 12B is a sectional configuration view of the lens of the objective optical system for an endoscope in the magnified observation state according to Example 6.

The objective optical system for an endoscope includes, in order from an object side: a first group G1 having a positive refractive power; an aperture stop S; a second group G2 having a negative refractive power; and a third group G3 having a positive refractive power.

The first group G1 having a positive refractive power includes, in order from the object side: a plano-concave negative lens L1 with a flat surface directed to the object side; a biconcave negative lens L2; a biconvex positive lens L3; an infrared absorbing filter F1; a plano-convex positive lens L4 with a flat surface directed to the object side; and a negative meniscus lens L5 with a convex surface directed to an image. The negative lens L2 and the positive lens L3 are cemented to forma cemented lens CL1. The positive lens L4 and the negative meniscus lens L5 are cemented to form a cemented lens CL2. The aperture stop S is disposed on the image side of the first group G1.

The second group G2 having a negative refractive power includes, in order from the object side: a negative meniscus lens L6 with a convex surface directed to the object side; and a positive meniscus lens L7 with a convex surface directed to the object side. The negative meniscus lens L6 and the positive meniscus lens L7 are cemented. The second group G2 moves toward the image along the optical axis AX at a time of focusing from the normal observation state to the magnified observation state. r12 and r16 are virtual planes.

The third group G3 having a positive refractive power includes, in order from the object side: a biconvex positive lens L8; a plano-convex positive lens L9 with a flat surface directed to the image; a biconvex positive lens L10; a biconcave negative lens L11; and a plano-convex positive lens L12 with a flat surface directed to the image. The positive lens L10 and the negative lens L11 are cemented to form a cemented lens CL3. The positive lens L12 and a cover glass CG are cemented. The cover glass CG being a parallel plate is cemented to an image plane I that is the image pickup surface of an image sensor not illustrated.

The infrared absorbing filter F1 being a parallel plate is a filter provided with a coating for cutting a specific wavelength, for example, 1060 nm of a YAG laser, 810 nm of a semiconductor laser, or an infrared band.

FIG. 13A illustrates spherical aberration (SA) in the normal observation state, FIG. 13B illustrates astigmatism (AS) in the normal observation state, FIG. 13C illustrates distortion (DT) in the normal observation state, and FIG. 13D illustrates a chromatic aberration of magnification (CC) in the normal observation state, for the objective optical system for an endoscope according to Example 6.

FIG. 13E illustrates spherical aberration (SA) in the magnified observation state, FIG. 13F illustrates astigmatism (AS) in the magnified observation state, FIG. 13G illustrates distortion (DT) in the magnified observation state, and FIG. 13H illustrates a chromatic aberration of magnification (CC) in the magnified observation state, for the objective optical system for an endoscope according to Example 6.

### Example 7

An objective optical system for an endoscope according to Example 7 will be explained hereinafter.

FIG. 14A is a sectional configuration view of a lens of an objective optical system for an endoscope in the normal observation state according to Example 7. FIG. 14B is a sectional configuration view of the lens of the objective optical system for an endoscope in the magnified observation state according to Example 7.

The objective optical system for an endoscope includes, in order from an object side: a first group G1 having a positive refractive power; an aperture stop S; a second group G2 having a negative refractive power; and a third group G3 having a positive refractive power.

The first group G1 having a positive refractive power includes, in order from the object side: a plano-concave negative lens L1 with a flat surface directed to the object side; a biconcave negative lens L2; a biconvex positive lens L3; an infrared absorbing filter F1; a biconvex positive lens L4; and a negative meniscus lens L5 with a convex surface directed to an image. The negative lens L2 and the positive lens L3 are cemented to form a cemented lens CL1. The positive lens L4 and the negative meniscus lens L5 are cemented to form a cemented lens CL2. The aperture stop S is disposed on the image side of the first group G1.

The second group G2 having a negative refractive power includes, in order from the object side: a negative meniscus lens L6 with a convex surface directed to the object side; and a positive meniscus lens L7 with a convex surface directed to the object side. The negative meniscus lens L6 and the positive meniscus lens L7 are cemented. The second group G2 moves toward the image along the optical axis AX at a time of focusing from the normal observation state to the magnified observation state. r12 and r16 are virtual planes.

The third group G3 having a positive refractive power includes, in order from the object side: a biconvex positive lens L8; a plano-convex positive lens L9 with a flat surface directed to the image; a biconvex positive lens L10; a biconcave negative lens L11; and a plano-convex positive lens L12 with a flat surface directed to the image. The positive lens L10 and the negative lens L11 are cemented to form a cemented lens CL3. The positive lens L12 and a cover glass CG are cemented. The cover glass CG being a parallel plate is cemented to an image plane I that is the image pickup surface of an image sensor not illustrated.

The infrared absorbing filter F1 being a parallel plate is a filter provided with a coating for cutting a specific wavelength, for example, 1060 nm of a YAG laser, 810 nm of a semiconductor laser, or an infrared band.

FIG. 15A illustrates spherical aberration (SA) in the normal observation state, FIG. 15B illustrates astigmatism (AS) in the normal observation state, FIG. 15C illustrates distortion (DT) in the normal observation state, and FIG. 15D illustrates a chromatic aberration of magnification (CC) in the normal observation state, for the objective optical system for an endoscope according to Example 7.

FIG. 15E illustrates spherical aberration (SA) in the magnified observation state, FIG. 15F illustrates astigmatism (AS) in the magnified observation state, FIG. 15G illustrates distortion (DT) in the magnified observation state, and FIG. 15H illustrates a chromatic aberration of magnification (CC) in the magnified observation state, for the objective optical system for an endoscope according to Example 7.

### Example 8

An objective optical system for an endoscope according to Example 8 will be explained hereinafter.

FIG. 16A is a sectional configuration view of a lens of an objective optical system for an endoscope in the normal observation state according to Example 8. FIG. 16B is a sectional configuration view of the lens of the objective optical system for an endoscope in the magnified observation state according to Example 8.

The objective optical system for an endoscope includes, in order from an object side: a first group G1 having a positive refractive power; an aperture stop S; a second group G2 having a negative refractive power; and a third group G3 having a positive refractive power.

The first group G1 having a positive refractive power includes, in order from the object side: a plano-concave negative lens L1 with a flat surface directed to the object side; a biconcave negative lens L2; a biconvex positive lens L3; an infrared absorbing filter F1; a positive meniscus lens L4 with a convex surface directed to the image; and a negative meniscus lens L5 with a convex surface directed to the image. The negative lens L2 and the positive lens L3 are cemented to form a cemented lens CL1. The positive meniscus lens L4 and the negative meniscus lens L5 are cemented to forma cemented lens CL2. The aperture stop S is disposed on the image side of the first group G1.

The second group G2 having a negative refractive power includes, in order from the object side: a negative meniscus lens L6 with a convex surface directed to the object side; and a positive meniscus lens L7 with a convex surface directed to the object side. The negative meniscus lens L6 and the positive meniscus lens L7 are cemented. The second group G2 moves toward the image along the optical axis AX at a time of focusing from the normal observation state to the magnified observation state. r12 and r16 are virtual planes.

The third group G3 having a positive refractive power includes, in order from the object side: a biconvex positive lens L8; a plano-convex positive lens L9 with a flat surface directed to the image; a biconvex positive lens L10; and a biconcave negative lens L11. The positive lens L10 and the negative lens L11 are cemented to form a cemented lens CL3. A parallel plate F2 and a cover glass CG are cemented. The cover glass CG being a parallel plate is cemented to an image plane I that is the image pickup surface of an image sensor not illustrated.

The infrared absorbing filter F1 being a parallel plate is a filter provided with a coating for cutting a specific wavelength, for example, 1060 nm of a YAG laser, 810 nm of a semiconductor laser, or an infrared band.

FIG. 17A illustrates spherical aberration (SA) in the normal observation state, FIG. 17B illustrates astigmatism (AS) in the normal observation state, FIG. 17C illustrates distortion (DT) in the normal observation state, and FIG. 17D illustrates a chromatic aberration of magnification (CC) in the normal observation state, for the objective optical system for an endoscope according to Example 8.

FIG. 17E illustrates spherical aberration (SA) in the magnified observation state, FIG. 17F illustrates astigmatism (AS) in the magnified observation state, FIG. 17G illustrates distortion (DT) in the magnified observation state, and FIG. 17H illustrates a chromatic aberration of magnification (CC) in the magnified observation state, for the objective optical system for an endoscope according to Example 8.

Examples of numerical values will be listed hereinafter. r1, r2, . . . denote a radius of curvature of each lens surface. d1, d2, . . . denote a thickness or surface distance of each lens. n1, n2, . . . denote a refractive index at the e-line for each lens. ν1, ν2, . . . denote Abbe's number at the d-line for each lens. Stop is an aperture stop.

### Example 1

Unit mm
Surface data

| Surface no. | r | d | ne | νe |
|---|---|---|---|---|
| 1 | ∞ | 0.51 | 1.88300 | 40.78 |
| 2 | 2.178 | 1.45 | | |
| 3 | −6.771 | 1.69 | 1.88300 | 40.76 |
| 4 | 2.096 | 2.41 | 1.51742 | 52.43 |
| 5 | −3.390 | 0.11 | | |
| 6 | ∞ | 0.89 | 1.49400 | 75.00 |
| 7 | ∞ | 0.22 | | |
| 8 | ∞ | 1.76 | 1.72916 | 54.68 |
| 9 | −3.216 | 1.14 | 1.84666 | 23.78 |
| 10 | −4.693 | 0.11 | | |
| 11(Stop) | ∞ | 0.07 | | |
| 12 | ∞ | Variable | | |
| 13 | ∞ | 0.71 | 1.49700 | 81.54 |
| 14 | 3.713 | 0.73 | 1.84666 | 23.78 |
| 15 | 4.212 | 0.30 | | |
| 16 | ∞ | Variable | | |
| 17 | 7.394 | 1.56 | 1.48749 | 70.23 |
| 18 | −7.394 | 0.22 | | |
| 19 | 4.194 | 1.34 | 1.53775 | 74.70 |
| 20 | ∞ | 0.29 | | |
| 21 | 4.563 | 1.14 | 1.51633 | 64.14 |
| 22 | −6.477 | 0.67 | 1.95906 | 17.47 |
| 23 | 3.780 | 0.77 | | |
| 24 | 4.009 | 1.00 | 1.51633 | 64.14 |
| 25 | ∞ | 0.02 | 1.51500 | 64.00 |
| 26 | ∞ | 0.78 | 1.50700 | 63.26 |
| 27 | ∞ | | | |
| 29(Image plane) | | | | |

Zoom data

| | Normal observation state | Magnified observation state |
|---|---|---|
| focal length | 1.05 | 1.25 |
| FNO. | 3.04 | 3.62 |
| d12 | 0.29 | 2.34 |
| d16 | 2.38 | 0.33 |

### Example 2

Unit mm
Surface data

| Surface no. | r | d | ne | νe |
|---|---|---|---|---|
| 1 | ∞ | 0.51 | 1.88300 | 40.78 |
| 2 | 2.895 | 1.45 | | |
| 3 | −5.431 | 1.70 | 1.88300 | 40.76 |
| 4 | 2.034 | 2.41 | 1.51742 | 52.43 |
| 5 | −3.389 | 0.26 | | |
| 6 | ∞ | 0.89 | 1.49400 | 75.00 |
| 7 | ∞ | 0.22 | | |
| 8 | 684.692 | 1.76 | 1.72916 | 54.68 |
| 9 | −3.212 | 1.14 | 1.84666 | 23.78 |
| 10 | −4.701 | 0.11 | | |
| 11(Stop) | ∞ | 0.07 | | |
| 12 | ∞ | Variable | | |
| 13 | 80.180 | 0.64 | 1.49700 | 81.54 |
| 14 | 3.722 | 0.61 | 1.84666 | 23.78 |
| 15 | 4.192 | 0.30 | | |
| 16 | ∞ | Variable | | |
| 17 | 7.394 | 1.56 | 1.48749 | 70.23 |
| 18 | −7.386 | 0.22 | | |
| 19 | 4.162 | 1.32 | 1.53775 | 74.70 |
| 20 | −70.480 | 0.29 | | |
| 21 | 4.548 | 1.12 | 1.51633 | 64.14 |
| 22 | −6.142 | 0.66 | 1.95906 | 17.47 |
| 23 | 3.795 | 0.49 | | |
| 24 | 4.009 | 1.00 | 1.51633 | 64.14 |
| 25 | ∞ | 0.02 | 1.51500 | 64.00 |
| 26 | ∞ | 0.78 | 1.50700 | 63.26 |
| 27 | ∞ | | | |
| 28(Image plane) | | | | |

Zoom data

| | Normal observation state | Magnified observation state |
|---|---|---|
| focal length | 1.08 | 1.27 |
| FNO. | 2.83 | 3.33 |
| d12 | 0.29 | 2.36 |
| d16 | 2.40 | 0.32 |

### Example 3

Unit mm
Surface data

| Surface no. | r | d | ne | νe |
|---|---|---|---|---|
| 1 | ∞ | 0.51 | 1.88300 | 40.78 |
| 2 | 2.218 | 1.45 | | |
| 3 | −5.859 | 1.64 | 1.88300 | 40.76 |
| 4 | 2.089 | 2.17 | 1.51742 | 52.43 |
| 5 | −3.575 | 0.13 | | |
| 6 | ∞ | 0.89 | 1.49400 | 75.00 |
| 7 | ∞ | 0.22 | | |
| 8 | 21.053 | 1.71 | 1.72916 | 54.68 |
| 9 | −3.206 | 1.08 | 1.84666 | 23.78 |
| 10 | −4.658 | 0.11 | | |
| 11(Stop) | ∞ | 0.07 | | |
| 12 | ∞ | Variable | | |
| 13 | 59.032 | 0.58 | 1.49700 | 81.54 |
| 14 | 3.812 | 0.52 | 1.84666 | 23.78 |
| 15 | 4.012 | 0.30 | | |
| 16 | ∞ | Variable | | |
| 17 | 7.765 | 1.62 | 1.48749 | 70.23 |
| 18 | −7.745 | 0.22 | | |
| 19 | 5.420 | 1.35 | 1.53775 | 74.70 |
| 20 | −43.839 | 0.29 | | |
| 21 | 4.518 | 1.24 | 1.51633 | 64.14 |
| 22 | −20.974 | 0.76 | 1.95906 | 17.47 |
| 23 | 3.746 | 0.07 | | |
| 24 | ∞ | 0.53 | | |
| 25 | 4.009 | 1.00 | 1.51633 | 64.14 |
| 26 | ∞ | 0.02 | 1.51500 | 64.00 |
| 27 | ∞ | 0.78 | 1.50700 | 63.26 |
| 28 | ∞ | | | |
| 29(Image plane) | | | | |

Zoom data

| | Normal observation state | Magnified observation state |
|---|---|---|
| focal length | 1.06 | 1.26 |
| FNO. | 2.94 | 3.49 |
| d12 | 0.26 | 2.25 |
| d16 | 2.27 | 0.27 |

### Example 4

Unit mm
Surface data

| Surface no. | r | d | ne | νe |
|---|---|---|---|---|
| 1 | ∞ | 0.51 | 1.88300 | 40.78 |
| 2 | 3.341 | 1.40 | | |
| 3 | −5.655 | 1.71 | 1.88300 | 40.76 |
| 4 | 2.020 | 2.42 | 1.51742 | 52.43 |
| 5 | −3.377 | 0.21 | | |
| 6 | ∞ | 0.89 | 1.49400 | 75.00 |
| 7 | ∞ | 0.52 | | |
| 8 | −142.693 | 1.76 | 1.72916 | 54.68 |
| 9 | −3.204 | 1.13 | 1.84666 | 23.78 |
| 10 | −4.707 | 0.00 | | |
| 11(Stop) | ∞ | 0.06 | | |
| 12 | ∞ | Variable | | |
| 13 | 54.409 | 0.66 | 1.49700 | 81.54 |
| 14 | 3.722 | 0.65 | 1.84666 | 23.78 |
| 15 | 4.197 | 0.06 | | |
| 16 | ∞ | Variable | | |
| 17 | 7.383 | 1.55 | 1.48749 | 70.23 |
| 18 | −7.373 | 0.10 | | |
| 19 | 3.827 | 1.34 | 1.53775 | 74.70 |
| 20 | ∞ | 0.26 | | |
| 21 | 4.548 | 1.15 | 1.51633 | 64.14 |
| 22 | −5.846 | 0.66 | 1.95906 | 17.47 |
| 23 | 3.786 | 0.09 | | |
| 24 | ∞ | 0.38 | | |
| 25 | 3.706 | 0.87 | 1.51633 | 64.14 |
| 26 | ∞ | 0.07 | 1.51500 | 64.00 |
| 27 | ∞ | 0.65 | 1.50700 | 63.26 |
| 28 | ∞ | | | |
| 29(Image plane) | | | | |

Zoom data

| | Normal observation state | Magnified observation state |
|---|---|---|
| focal length | 1.09 | 1.28 |
| FNO. | 2.72 | 3.18 |
| d12 | 0.26 | 2.36 |
| d16 | 2.40 | 0.30 |

### Example 5

Unit mm
Surface data

| Surface no. | r | d | ne | νe |
|---|---|---|---|---|
| 1 | ∞ | 1.46 | 1.88300 | 40.78 |
| 2 | 2.288 | 1.45 | | |
| 3 | −6.192 | 1.64 | 1.88300 | 40.76 |
| 4 | 2.088 | 2.38 | 1.51742 | 52.43 |
| 5 | −3.436 | 0.22 | | |
| 6 | ∞ | 0.89 | 1.49400 | 75.00 |
| 7 | ∞ | 0.16 | | |
| 8 | 844.855 | 1.76 | 1.72916 | 54.68 |
| 9 | −3.247 | 1.14 | 1.84666 | 23.78 |
| 10 | −4.655 | 0.33 | | |
| 11(Stop) | ∞ | 0.07 | | |
| 12 | ∞ | Variable | | |
| 13 | −147.787 | 0.67 | 1.49700 | 81.54 |
| 14 | 3.696 | 0.69 | 1.84666 | 23.78 |
| 15 | 4.226 | 0.96 | | |
| 16 | ∞ | Variable | | |
| 17 | 7.418 | 1.56 | 1.48749 | 70.23 |
| 18 | −7.436 | 0.55 | | |
| 19 | 3.805 | 1.33 | 1.53775 | 74.70 |
| 20 | ∞ | 0.00 | | |
| 21 | 4.584 | 1.11 | 1.51633 | 64.14 |
| 22 | −7.766 | 0.67 | 1.95906 | 17.47 |
| 23 | 3.775 | 0.64 | | |
| 24 | 7.557 | 1.12 | 1.51633 | 64.14 |
| 25 | ∞ | 0.14 | 1.51500 | 64.00 |
| 26 | ∞ | 0.90 | 1.50700 | 63.26 |
| 27 | ∞ | | | |
| 28(Image plane) | | | | |

Zoom data

| | Normal observation state | Magnified observation state |
|---|---|---|
| focal length | 1.07 | 1.27 |
| FNO. | 3.03 | 3.60 |
| d12 | 0.34 | 2.29 |
| d16 | 2.33 | 0.40 |

### Example 6

Unit mm
Surface data

| Surface no. | r | d | ne | νe |
|---|---|---|---|---|
| 1 | ∞ | 0.43 | 1.88300 | 40.78 |
| 2 | 2.166 | 1.47 | | |
| 3 | −9.759 | 1.70 | 1.88300 | 40.76 |
| 4 | 2.153 | 2.44 | 1.51742 | 52.43 |
| 5 | −3.319 | 0.22 | | |
| 6 | ∞ | 0.89 | 1.49400 | 75.00 |
| 7 | ∞ | 0.24 | | |
| 8 | −72.895 | 1.76 | 1.72916 | 54.68 |
| 9 | −3.213 | 1.13 | 1.84666 | 23.78 |
| 10 | −4.693 | 0.08 | | |
| 11(Stop) | ∞ | 0.07 | | |
| 12 | ∞ | Variable | | |
| 13 | 748.308 | 0.66 | 1.49700 | 81.54 |
| 14 | 3.715 | 0.66 | 1.84666 | 23.78 |
| 15 | 4.213 | 0.29 | | |
| 16 | ∞ | Variable | | |
| 17 | 7.392 | 1.56 | 1.48749 | 70.23 |
| 18 | −7.393 | 0.17 | | |
| 19 | 4.102 | 1.34 | 1.53775 | 74.70 |
| 20 | ∞ | 0.30 | | |
| 21 | 4.561 | 1.14 | 1.51633 | 64.14 |
| 22 | −6.708 | 0.67 | 1.95906 | 17.47 |
| 23 | 3.780 | 0.68 | | |
| 24 | 4.040 | 0.97 | 1.51633 | 64.14 |
| 25 | ∞ | 0.01 | 1.51500 | 64.00 |
| 26 | ∞ | 0.74 | 1.50700 | 63.26 |
| 27 | ∞ | | | |
| 28(Image plane) | | | | |

Zoom data

| | Normal observation state | Magnified observation state |
|---|---|---|
| focal length | 1.05 | 1.25 |
| FNO. | 2.92 | 3.47 |
| d12 | 0.28 | 2.34 |
| d16 | 2.38 | 0.33 |

### Example 7

Unit mm
Surface data

| Surface no. | r | d | ne | νe |
|---|---|---|---|---|
| 1 | ∞ | 0.60 | 1.88300 | 40.78 |
| 2 | 2.163 | 1.30 | | |
| 3 | −4.790 | 1.68 | 1.88300 | 40.76 |
| 4 | 2.108 | 2.40 | 1.51742 | 52.43 |
| 5 | −3.343 | 0.21 | | |
| 6 | ∞ | 0.89 | 1.49400 | 75.00 |
| 7 | ∞ | 0.22 | | |
| 8 | 103.811 | 1.75 | 1.72916 | 54.68 |
| 9 | −3.186 | 1.11 | 1.84666 | 23.78 |
| 10 | −4.731 | 0.22 | | |
| 11(Stop) | ∞ | 0.07 | | |
| 12 | ∞ | Variable | | |
| 13 | 371.937 | 0.67 | 1.49700 | 81.54 |
| 14 | 3.728 | 0.67 | 1.84666 | 23.78 |
| 15 | 4.208 | 0.35 | | |
| 16 | ∞ | Variable | | |
| 17 | 7.396 | 1.56 | 1.48749 | 70.23 |
| 18 | −7.401 | 0.31 | | |
| 19 | 4.763 | 1.34 | 1.53775 | 74.70 |
| 20 | ∞ | 0.62 | | |
| 21 | 4.561 | 1.14 | 1.51633 | 64.14 |
| 22 | −6.231 | 0.69 | 1.95906 | 17.47 |
| 23 | 3.786 | 0.76 | | |
| 24 | 3.536 | 0.89 | 1.51633 | 64.14 |
| 25 | ∞ | 0.02 | 1.51500 | 64.00 |
| 26 | ∞ | 0.78 | 1.50700 | 63.26 |
| 27 | ∞ | | | |
| 28(Image plane) | | | | |

Zoom data

| | Normal observation state | Magnified observation state |
|---|---|---|
| focal length | 1.05 | 1.26 |
| FNO. | 3.18 | 3.79 |
| d12 | 0.30 | 2.33 |
| d16 | 2.38 | 0.34 |

### Example 8

Unit mm
Surface data

| Surface no. | r | d | ne | νe |
|---|---|---|---|---|
| 1 | ∞ | 0.95 | 1.88300 | 40.78 |
| 2 | 2.252 | 1.25 | | |
| 3 | −7.573 | 1.79 | 1.88300 | 40.76 |
| 4 | 2.142 | 2.44 | 1.51742 | 52.43 |
| 5 | −3.233 | 0.23 | | |
| 6 | ∞ | 0.89 | 1.49400 | 75.00 |
| 7 | ∞ | 0.30 | | |
| 8 | −54.770 | 1.77 | 1.72916 | 54.68 |
| 9 | −3.212 | 1.17 | 1.84666 | 23.78 |
| 10 | −4.710 | 0.22 | | |
| 11(Stop) | ∞ | 0.07 | | |
| 12 | ∞ | Variable | | |
| 13 | 160.517 | 0.66 | 1.49700 | 81.54 |
| 14 | 3.671 | 0.61 | 1.84666 | 23.78 |
| 15 | 4.277 | 1.91 | | |
| 16 | ∞ | Variable | | |
| 17 | 7.351 | 1.57 | 1.48749 | 70.23 |
| 18 | −7.279 | 0.14 | | |
| 19 | 3.868 | 1.32 | 1.53775 | 74.70 |
| 20 | ∞ | 0.05 | | |
| 21 | 4.350 | 1.22 | 1.51633 | 64.14 |
| 22 | −6.167 | 0.67 | 1.95906 | 17.47 |
| 23 | 4.008 | 0.64 | | |
| 24 | ∞ | 1.00 | 1.51633 | 64.14 |
| 25 | ∞ | 0.02 | 1.51500 | 64.00 |
| 26 | ∞ | 0.78 | 1.50700 | 63.26 |
| 27 | ∞ | | | |
| 28(Image plane) | | | | |

Zoom data

| | Normal observation state | Magnified observation state |
|---|---|---|
| focal length | 1.06 | 1.26 |
| FNO. | 2.91 | 3.47 |
| d12 | 0.21 | 2.41 |
| d16 | 2.50 | 0.22 |

Corresponding values of the conditional expression of each examples are shown below.

| | Example 1 | Example 2 | Example 3 | Example 4 |
|---|---|---|---|---|
| (1) | −2.33 | −3.03 | −2.36 | −3.46 |
| (2) | −1.42 | −1.44 | −1.07 | −1.47 |
| (3) | −1.41 | −1.35 | −1.18 | −1.33 |
| (4) | 2.66 | 4.18 | 3.33 | 4.42 |
| (5) | 1.36 | 1.53 | 1.36 | 1.62 |
| (6) | −0.82 | −0.81 | −1.34 | −0.77 |
| (7) | −12.39 | −8.84 | −8.30 | −9.18 |
| (8) | −1.93 | −1.41 | −1.54 | −1.44 |
| (9) | −3.15 | −2.37 | −3.10 | −1.90 |
| (10) | 0.26 | 0.24 | 0.70 | 0.21 |
| | Example 5 | Example 6 | Example 7 | Example 8 |
| (1) | −2.41 | −2.32 | −2.31 | −2.39 |
| (2) | −1.35 | −1.40 | −1.44 | −1.26 |
| (3) | −1.21 | −1.36 | −1.62 | −1.16 |
| (4) | 3.04 | 3.04 | 2.68 | 3.45 |
| (5) | 1.77 | 1.36 | 1.35 | 1.55 |
| (6) | −0.88 | −0.79 | −0.83 | −0.85 |
| (7) | −10.30 | −25.43 | −8.45 | −18.83 |
| (8) | −1.66 | −3.77 | −1.35 | −2.76 |
| (9) | −5.66 | −3.20 | −2.80 | −0.87 |
| (10) | 0.35 | 0.28 | 0.24 | 0.21 |

As described above, the present disclosure is suitable for a compact and high-definition objective optical system for an endoscope for which the deterioration of an optical performance due to a manufacturing error is reduced, an image pickup apparatus, and an endoscope.

According to the present disclosure, it is possible to provide a compact and high-definition objective optical system for an endoscope for which the deterioration of an optical performance due to a manufacturing error is reduced, an image pickup apparatus, and an endoscope.

What is claimed is:

1. An objective optical system for an endoscope comprising, in order from an object side:
a first group having a positive refractive power;
a second group having a negative refractive power; and
a third group having a positive refractive power, wherein
at least the second group is moved along an optical axis to change magnification and perform focusing under a normal observation state and a magnified observation state,
the first group includes, in order from the object side: a plano-concave negative lens with a flat surface directed to the object side; and two cemented lenses, and
a following conditional expression (1) is satisfied:
−3.6<f1/fz1<−2 (1)
where
f1 denotes a focal length of the plano-concave negative lens, and
fz1 denotes a focal length of the entire objective optical system for an endoscope in the normal observation state.

2. The objective optical system for an endoscope according to claim 1, wherein
the third group includes, in order from the object side:
a positive lens;
a positive lens; and
a cemented lens in which a positive lens and a negative lens are cemented, and
following conditional expressions (2) and (3) are satisfied:
−5<f5/f7<−1 (2)
−5<f6/f7<−0.3 (3)
where
f5 denotes a focal length of the object-side positive lens of the third group,
f6 denotes a focal length of the image-side positive lens of the third group, and
f7 denotes a focal length of the cemented lens of the third group.

3. The objective optical system for an endoscope according to claim 1, wherein a following conditional expression (4) is satisfied:
2.1<Ls/Bk<5 (4)
where
Ls denotes a movement distance of the second group from the normal observation state to the magnified observation state, and
Bk denotes a distance from the final surface of the objective optical system for an endoscope to an image plane along an optical axis.

4. The objective optical system for an endoscope according to claim 1, wherein a following conditional expression (5) is satisfied:
0.8<FFz3/fz3<4 (5)
where
FFz3 denotes a distance from the front focal point of the objective optical system for an endoscope in the magnified observation state to the surface of the objective optical system for an endoscope positioned nearest to the object, and
fz3 denotes a focal length of the entire objective optical system for an endoscope in the magnified observation state.

5. The objective optical system for an endoscope according to claim 2, wherein a following conditional expression (6) is satisfied:
−6<f7/f3<−0.5 (6)
f7 denotes a focal length of the cemented lens of the third group, and
f3 denotes a focal length of the image-side cemented lens of the first group.

6. The objective optical system for an endoscope according to claim 1, wherein a following conditional expression (7) is satisfied:
−30<f2/fz1<−1 (7)
where
f2 denotes a focal length of the object-side cemented lens of the first group, and
fz1 denotes the focal length of the entire objective optical system for an endoscope in the normal observation state.

7. The objective optical system for an endoscope according to claim 1, wherein a following conditional expression (8) is satisfied:
−8<f2/f3<−1 (8)
where
f2 denotes a focal length of the object-side cemented lens of the first group, and
f3 denotes a focal length of the image-side cemented lens of the first group.

8. The objective optical system for an endoscope according to claim 1, wherein
the third group further includes a plano-convex positive lens with a flat surface cemented to a cover glass and directed to an image plane, and
a following conditional expression (9) is satisfied:
−10<f8/f1<−0.5 (9)
where
f8 denotes a focal length of the positive lens cemented to the cover glass, and
f1 denotes the focal length of the plano-concave negative lens.

9. The objective optical system for an endoscope according to claim 1, wherein
the cemented lens of the third group further includes a biconcave negative lens, and
a following conditional expression (10) is satisfied:
0.1<SF72<0.9 (10)
where
SF72 denotes a shaping factor of the biconcave negative lens, and when r72 is a radius of the object-side curvature of the biconcave negative lens and r73 is a radius of the image-side curvature of the biconcave negative lens, SF72=(r72+r73)/(r72−r73).

10. An image pickup apparatus comprising the objective optical system for an endoscope according to claim 1.

11. An endoscope comprising the objective optical system for an endoscope according to claim 1.
